(19) United States
(12) Patent Application Publication
(10) Pub. No.: US 2021/0169823 A1
(43) Pub. Date: Jun. 10, 2021

(54) TOPICAL SKIN CARE COMPOSITION AND METHODS FOR TREATING ECZEMA

(71) Applicant: Avicanna Inc., Toronto (CA)

(72) Inventors: Frantz Henri Emmanuel LE DEVEDEC, North York (CA); Aras AZADIAN, Toronto (CA); Setu Nimish PUROHIT, Toronto (CA); Justin Michael GRANT, Toronto (CA); Samantha Carolyn WATT, Toronto (CA)

(73) Assignee: Avicanna Inc., Toronto (CA)

(21) Appl. No.: 16/709,489

(22) Filed: Dec. 10, 2019

(51) Int. Cl. A61K 31/05; A61K 8/49; A61K 8/34; A61K 8/04; A61K 8/06; A61Q 19/00; A61K 9/00; A61K 31/352; A61P 17/00

(52) U.S. Cl. CPC A61K 31/05; A61K 8/498; A61K 8/347; A61K 8/046; A61P 17/00; A61K 8/06; A61Q 19/007; A61K 9/0014; A61K 31/352; A61K 8/042

(57) ABSTRACT

Topical composition comprising cannabinoids in combination with one or more skin conditioning agents, one or more skin soothing agents, one or more occlusives and one or more emulsifiers are provided. Also provided are methods comprising applying a topical composition provided herein to the skin of a subject and uses of a topical composition for enhancing skin moisture content and for reducing skin moisture evaporation in treating and preventing eczema or any symptom or side effect associated with it.

### FIELD

The present disclosure relates to topical skin care composition and methods for moisturizing and hydrating eczematous skin.

### BACKGROUND

The skin provides the body with mechanical protection and acts as a chemical barrier to restrict foreign substance penetration, to prevent water or fluid loss and to maintain a constant temperature. Dry skin is a common condition that affects almost all of people at some time in their lives. Dry skin can cause many skin problems including atopic dermatitis, eczema, psoriasis and pruritus.

There is consequently ongoing research to introduce into cosmetic or dermatological industry new compositions which prevents dryness and deterioration of the skin. Several topical compositions are currently available. These compositions have various drawbacks ranging from unpleasant tactile properties (e.g., heavy, greasy, or sticky feel), instability issues, skin-irritation issues, insufficient moisturization capabilities, or the compositions are too harsh for sensitive skin.

There is a growing demand among the consumers of natural remedies as alternative or complementary therapy to improve general skin health and to treat skin diseases. Additionally, there is a particular need to treat the symptoms of skin diseases, with a specific focus on alleviating itch, redness and reducing inflammation in the course of the skin diseases through topical application of active natural agents. In particular, such active natural agents applied topically should exert less negative side effects than chemically synthesized agents.

There is also a need to use natural remedies to restore skin's appearance, physical properties, or physiological functions. Thus, a product that has no or fewer adverse effects and does not cause skin irritation would be advantageous. With relaxation of laws regulating cannabis use, there now exists the opportunity to explore the potential of phytocannabinoids to address such skin problems.

### SUMMARY

In one aspect, there is provided a topical skin care composition comprising:
a. a cannabinoid at 0.01-10% (w/w),
b. a skin conditioning agent at 0.01-5% (w/w),
c. a skin soothing agent at 1-5% (w/w),
d. an occlusive at 0.01-5% (w/w),
e. an emulsifier at 1-10% (w/w),
f. water to make up 100% by weight.
   wherein the said topical skin care composition comprises less than 20% (w/w) of fatty alcohol.

In an embodiment of the topical skin care composition as described herein, the topical skin care composition comprises no more than 10% (w/w) of fatty alcohol.

In an embodiment of the topical skin care composition as described herein, the topical skin care composition comprises no more than 5% (w/w) of fatty alcohol.

In another aspect, there is provided a topical skin care composition comprising:
a. a cannabinoid at 0.01-10% (w/w),
b. a skin conditioning agent at 0.01-5% (w/w),
c. a skin soothing agent at 1-5% (w/w),
d. an occlusive at 0.01-5% (w/w),
e. an emulsifier at 1-10% (w/w),
f. water no less than 51% by weight.

In an embodiment of the topical skin care composition as described herein, the cannabinoid is a phytocannabinoid.

In an embodiment of the topical skin care composition as described herein, the cannabinoid is tetrahydrocannabinol (THC), tetrahydrocannabinolic acid (THCA), cannabidiol (CBD), cannabidiolic acid (CBDA), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigervarin (CBGV), cannabigerol monomethyl ether (CBGM), cannabielsoin (CBE), cannabicitran (CBT), or any combination thereof.

In an embodiment of the topical skin care composition as described herein, the cannabinoid is cannabidiol (CBD).

In an embodiment of the topical skin care composition as described herein, the skin conditioning agent is colloidal oatmeal, the skin soothing agent is oat kernel extract, the occlusive is hemp seed oil and the emulsifier is glyceryl stearate.

In an embodiment of the topical skin care composition as described herein, the composition has a pH of 5-7.

In an embodiment of the topical skin care composition as described herein, the composition further comprises one or more of:
a. a first surfactant at 1-10% (w/w),
b. a second surfactant at 1-5% (w/w),
c. a first emollient at 1-15% (w/w),
d. a second emollient at 0.01-5% (w/w),
e. a humectant at 0.01-5% (w/w),
f. a penetration enhancer at 1-10% (w/w),
g. an opacifying agent at 0.01-3% (w/w),
h. a stabilizer at 0.01-5% (w/w),
i. a preservative at 0.01-3% (w/w),
j. pH adjusting agents in a quantity sufficient for the composition to maintain a pH of 5-7, and
k. water to make up 100% by weight.

In an embodiment of the topical skin care composition as described herein, the first surfactant is cetostearyl alcohol, the second surfactant is Tween 60, the first emollient is white petrolatum, the second emollient is dimethicone, the humectant is glycerin, the penetration enhancer is ethoxydiglycol, the opacifying agent is titanium (IV) oxide, the stabilizer is disodium salt of ethylenediaminetetraacetic acid (EDTA), the preservative is phenoxyethanol SA, pH adjusting agents are trisodium citrate dihydrate and citric acid monohydrate.

In an embodiment of the topical skin care composition as described herein, the composition is a cream, ointment, gel, lotion, liquid, solution, spray, aerosol, any other dosage forms suitable for topical application, or any combination thereof.

In an embodiment of the topical skin care composition as described herein, the composition is a lotion.

In another aspect, there is provided a lotion comprising:
a. an antioxidant at 0.01-10% (w/w),
b. a skin conditioning agent at 0.01-5% (w/w),
c. an emulsifier at 1-10% (w/w),
d. an occlusive at 0.01-5% (w/w), and
e. water to make up 100% by weight.

In an embodiment of the lotion as described herein, the composition has a viscosity in a range of about 5,000 cps to about 100,000 cps.

In another aspect, there is provided a method comprising applying the topical skin care composition as described herein to the skin of a subject.

In an embodiment of the method described herein, the skin of the subject is affected by a skin disease or condition.

In an embodiment of the method described herein, the skin disease or condition is: contact eczema/dermatitis, atopic eczema/dermatitis, seborrheic eczema/dermatitis, stasis eczema/dermatitis, nummular eczema/dermatitis, lichen simplex chronicus, dyshidrotic eczema/dermatitis, erythema, irritation, pruritus, dried skin, cracked skin, wrinkles and rosacea.

In another aspect, there is provided use of the topical skin care composition described herein for the treatment of a skin disease or condition in a subject.

In an embodiment of the use as described herein, the skin disease or condition is: contact eczema/dermatitis, atopic eczema/dermatitis, seborrheic eczema/dermatitis, stasis eczema/dermatitis, nummular eczema/dermatitis, lichen simplex chronicus, dyshidrotic eczema/dermatitis, erythema, irritation, pruritus, dried skin, cracked skin, wrinkles and rosacea.

Other aspects, features, and embodiments of the present disclosure will become apparent to those of ordinary skill in the art upon review of the following description of specific embodiments.

### BRIEF DESCRIPTION OF THE DRAWINGS

FIG. 1 illustrates capacitance measured at baseline, as well as at 1 and 3 hours after single application of composition versus control.

FIG. 2 illustrates percentage (%) change in capacitance measured at baseline, as well as at 1 and 3 hours after single application of composition versus control.

FIG. 3 illustrates results of Welch two sample t-test on capacitance measurements between the composition and control at 1 hour (left, p<0.001) and at 3 hours (right, p=0.008) after single application.

FIG. 4 illustrates 95% family-wise confidence level for % capacitance change at 1 and 3 hours after single application of composition.

FIG. 5 illustrates capacitance measured at baseline, as well as at visit 1 (2 weeks) and visit 2 (4 weeks) after repeated application of composition versus control.

FIG. 6 illustrates percentage (%) change in capacitance measured at baseline, as well as at visit 1 (2 weeks) and visit 2 (4 weeks) after repeated application of composition versus control.

FIG. 7 illustrates comparison of capacitance measured at baseline and visit 2 (2 weeks) of composition versus control.

FIG. 8 illustrates 95% family-wise confidence level for % capacitance change at visit 1 (2 weeks) and visit 2 (4 weeks) after repeated application of composition.

FIG. 9 illustrates mean erythema values on volar forearm in subjects treated with the composition versus control.

FIG. 10 illustrates significant reduction in facial erythema values in subjects treated with the composition.

### DETAILED DESCRIPTION

It has been surprisingly discovered by the present inventors that certain extracts of Cannabis plant species when combined with colloidal oatmeal, oat kernel extract and hemp seed oil showed unexpected synergy in providing superior skin hydration properties, reducing the signs of erythema and reducing skin inflammation.

The present invention relates to compositions comprising plant extracts for use on skin. More specifically, the present invention relates to compositions comprising extracts of Cannabis plant species for improving the condition and appearance of the skin, such as by improving skin barrier protection, improving hydration and moisturization of the skin and reducing inflammation of the skin.

There is provided herein a topical skin care composition useful for enhancing skin moisture content and for reducing skin moisture evaporation in treating and preventing eczema or any symptom or side effect associated with it. Non-limiting examples of symptoms include erythema or redness in the affected skin area; pruritus or itching in the affected skin area; exudation or oozing and/or crusting in the affected skin area; excoriation or evidence of scratching in the affected skin area; and lichenification or epidermal thickening in the affected skin area. The topical skin care composition maintains a moist skin environment by forming a protective barrier to benefit the healing process of the damaged or affected skin.

In an aspect, the composition can be applied directly to the eczematous skin and can help reduce itch, inflammation, dryness and redness. Composition can also provide a soothing effect at the site of application to the skin.

Composition provided herein can also be used in conjunction with available treatments of skin diseases. Composition can also be used for routine dermatological purposes for the prevention of itch, skin dryness and redness.

Topical skin care composition provided herein exhibit excellent overall stability and viscosity.

“Cannabinoid,” as used herein, is meant to include compounds which interact with the cannabinoid receptor and various cannabinoid mimetics, such as tetrahydrocannabinol (THC), tetrahydrocannabinolic acid (THCA), cannabidiol (CBD), cannabidiolic acid (CBDA), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigervarin (CBGV), cannabigerol monomethyl ether (CBGM), cannabielsoin (CBE) and cannabicitran (CBT).

“Phytocannabinoid” as used herein means cannabinoid extracted from Cannabis plant species including by the way of non-limiting example Cannabis sativa, Cannabis indica and Cannabis ruderalis and all resins, stalks, flowers, seeds and oils related thereto.

The terms “topical composition” or “topical formulation”, as used herein, means a composition may be placed for direct application to a skin surface and from which a therapeutically effective amount of the ingredients may be released. Such formulations may include creams, ointments, gels, lotions, or any other dosage form suitable for topical application.

The terms “skin” or “skin surface” is meant to include the outer skin of a subject comprising one or more epidermal layers.

The term “lotion” relates to a low- to medium-viscosity topical preparation intended for application to unbroken skin in contrast, creams and gels have higher viscosity. Lotions are applied to external skin with bare hands, a clean cloth, cotton wool or gauze. Many lotions, especially hand lotions and body lotions are formulated not as a medicine delivery system, but simply to smooth, re-hydrate, and soften the skin.

The terms “therapeutically effective amount” or “therapeutically and/or prophylactically effective amount” or “efficacy” as used herein, refers to an amount of compound or composition that is sufficient to elicit the required or desired therapeutic and/or prophylactic response, as the particular treatment context may require. It will be understood that a therapeutically and/or prophylactically effective amount of a compound or a composition for a subject is dependent inter alia on the body weight of the subject as well as other factors known to a person of ordinary skill in the art.

The term “ingredient” or “excipient” herein means any substance, which may be combined with an active agent or with other ingredients to improve handling or storage properties or to permit or facilitate formation of a dose unit of the composition. Examples of excipients include, but are not limited to, a “skin conditioning agent”, that softens, protects and/or cleanses the skin; a “skin soothing agent”, that treats sensitive or irritated skin by inhibiting inflammatory mediators; an “occlusive”, that prevents or obstruct something, in this case, a substance which prevents the removal of moisture (via evaporation) via film binding from the surface of the skin; an “emulsifier” or “emulsifying agent”, which is capable of lowering surface tension between a non-polar and polar phase; a “surfactant”, which act as solubilizing agent by lowering the surface tension (or interfacial tension) between two liquids or between a liquid and a solid; an “emollient”, which is capable of softening and soothing of the skin; a “humectant”, which is capable of attracting moisture; a “penetration enhancer”, which is capable of improving penetration of a composition into the epidermis; an “opacifying agent”, that is insoluble in the composition and that increases the opacity of the composition into which it is introduced; a stabilizer” or “stability agent”, which is added to a composition to improve its stability under conditions such as weekly cycling between freezer and ambient room temperature; a “preservative”, which prevents decomposition of composition by microbial growth or by undesirable chemical changes and a “pH adjusting agent/buffering agent”, which is capable of adjusting the pH (acidity or alkalinity) of the composition.

The term “skin hydration” refers to the amount of water in the stratum corneum, the outermost layer of the skin, and is often used as one of the measurements of skin health. General characteristics of healthy skin include a gradual increase in the water concentration of the stratum corneum. Dehydrated skin is often associated with poor barrier function, often resulting in reduced protection against disease causing organisms, toxins, and the environment.

“wt %” or “w/w %” when referring to the percentage of a component in a composition is percentage of the weight of the component in the composition relative to the total weight of the composition.

As used herein, the terms “stable” or “stability”, when referring to a composition, means that the composition can be cycled weekly between freezer and ambient room temperature conditions for a minimum of 1 month while retaining its pH and viscosity within defined ranges.

The term “viscosity” as used herein refers to the measure of the extent to which a fluid or an aqueous composition resists a force tending to cause it to flow.

The terms “treat,” “treating,” or “treatment of” are used herein in their broad senses unless otherwise specifically indicated in the particular context, and results of a treatment may generally include reversing, alleviating, or inhibiting the progress of an indicated disorder or condition, or one or more symptoms of the disorder or condition.

The term “prevention”, as used herein, refers to the ability of a substance or composition to prevent, delay or hinder the appearance or development of a disease, disorder, condition or change before its appearance.

“Alleviate” as used herein, is meant to include complete elimination as well as any clinically or quantitatively measurable reduction in the subject's symptoms and/or discomfort.

A “subject” herein to which a therapeutic agent or composition thereof can be administered includes mammals such as a human subject of either sex and of any age, and also includes any nonhuman animal, particularly a domestic or companion animal such as a cat, dog or horse, as well as laboratory animals such as guinea pigs.

As discussed in greater detail in the illustrative and non-limiting examples provided herein, the present disclosure is directed to topical skin care formulations/compositions for moisturizing and hydrating eczematous skin.

In one aspect, there is provided a topical skin care composition comprising:
a. a cannabinoid at 0.01-10% (w/w),
b. a skin conditioning agent at 0.01-5% (w/w),
c. a skin soothing agent at 1-5% (w/w),
d. an occlusive at 0.01-5% (w/w),
e. an emulsifier at 1-10% (w/w),
f. water to make up 100% by weight.
   wherein the topical skin care composition comprises less than 20% (w/w) of fatty alcohol. As used herein, the term “fatty alcohol” means aliphatic, monohydric, primary alcohol that has unbranched or branched hydrocarbon residues having 4 to 40 carbon atoms. As used herein, a concentration of a component referred herein is a total concentration when a topical skin care composition provided herein comprises two or more compounds that are considered to belong to the same component (e.g., for a topical skin care composition comprising a mixture of tetrahydrocannabinol and cannabidiol, both cannabinoids, 5% (w/w) of a cannabinoid is the total concentration of the mixture of tetrahydrocannabinol and cannabidiol).

In certain exemplary, non-limiting embodiments, a topical skin care composition provided herein comprises less than 15%, less than 10%, less than 5%, less than 1%, less than 0.5%, or less than 0.1% (w/w) of fatty alcohol.

In another aspect, there is provided a topical skin care composition comprising:
a. a cannabinoid at 0.01-10% (w/w),
b. a skin conditioning agent at 0.01-5% (w/w),
c. a skin soothing agent at 1-5% (w/w),
d. an occlusive at 0.01-5% (w/w),
e. an emulsifier at 1-10% (w/w),
f. water no less than 51% by weight.

In certain exemplary, non-limiting embodiments, a topical skin care composition provided herein comprises water at no less than 55% (w/w), 60% (w/w), 65% (w/w), 70% (w/w), 75% (w/w), 80% (w/w), 85% (w/w), 90% (w/w), 91% (w/w), 92% (w/w), 93% (w/w), 94% (w/w), 95% (w/w), 96% (w/w), 97% (w/w), or 98% (w/w).

In another aspect, there is provided a lotion comprising:
a. a cannabinoid at 0.01-10% (w/w),
b. a skin conditioning agent at 0.01-5% (w/w),
c. an emulsifying agent 1-10% (w/w),
d. an occlusive at 0.01-5% (w/w), and
e. water to make up 100% by weight.

Exemplary cannabinoids include tetrahydrocannabinol (THC), tetrahydrocannabinolic acid (THCA), cannabidiol (CBD), cannabidiolic acid (CBDA), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigervarin (CBGV), cannabigerol monomethyl ether (CBGM), cannabielsoin (CBE), cannabicitran (CBT), combinations, and mixtures thereof extracted from Cannabis plant species including Cannabis sativa, Cannabis indica and Cannabis ruderalis and all resins, stalks, flowers, seeds and oils related thereto.

In certain exemplary, non-limiting embodiments, a topical skin care composition provided herein may include about 0.1% to about 10% (w/w) of cannabinoid(s). For example, a topical skin care composition provided herein may comprise about 0.1% to about 10%, about 0.5% to about 10%, about 1% to about 10%, about 2% to about 10%, about 3% to about 10%, about 4% to about 10%, about 5% to about 10%, about 6% to about 10%, about 7% to about 10%, about 8% to about 10%, about 9% to about 10%, about 0.1% to about 9%, about 0.5% to about 9%, about 1% to about 9%, about 2% to about 9%, about 3% to about 9%, about 4% to about 9%, about 5% to about 9%, about 6% to about 9%, about 7% to about 9%, about 8% to about 9%, about 0.1% to about 8%, about 0.5% to about 8%, about 1% to about 8%, about 2% to about 8%, about 3% to about 8%, about 4% to about 8%, about 5% to about 8%, about 6% to about 8%, about 7% to about 8%, about 0.1% to about 7%, about 0.5% to about 7%, about 1% to about 7%, about 2% to about 7%, about 3% to about 7%, about 4% to about 7%, about 5% to about 7%, about 6% to about 7%, about 0.1% to about 6%, about 0.5% to about 6%, about 1% to about 6%, about 2% to about 6%, about 3% to about 6%, about 4% to about 6%, about 5% to about 6%, about 0.1% to about 5%, about 0.5% to about 5%, about 1% to about 5%, about 2% to about 5%, about 3% to about 5%, about 4% to about 5%, about 0.1% to about 4%, about 0.5% to about 4%, about 1% to about 4%, about 2% to about 4%, about 3% to about 4%, about 0.1% to about 3%, about 0.5% to about 3%, about 1% to about 3%, about 2% to about 3%, about 0.1% to about 2%, about 0.5% to about 2%, about 1% to about 2%, about 0.1% to about 1%, or about 0.5% to about 1% (w/w) of cannabinoid(s).

In certain exemplary, non-limiting embodiments, a topical skin care composition provided herein may comprise at least 0.1%, at least 0.5%, at least 1%, at least 2%, at least 3%, at least 4%, at least 5% (w/w), at least 6% (w/w), at least 7% (w/w), at least 8% (w/w), at least 9% (w/w) or at least 10% (w/w) of cannabinoid(s).

In certain exemplary, non-limiting embodiments, a topical skin care composition provided herein may comprise about 0.1%, about 0.5%, about 1%, about 2%, about 3%, about 4%, about 5%, about 6%, about 7%, about 8%, about 9% or about 10% (w/w) of cannabinoid(s).

In certain exemplary, non-limiting embodiments, topical skin care composition provided herein may be provided as cream, ointment, gel, liquid, lotion, solution, spray, aerosol, or combinations thereof.

In certain exemplary, non-limiting embodiments, a topical skin care composition provided herein may have a viscosity in a range of about 5,000 to about 100,000 cps.

In certain exemplary, non-limiting embodiments, topical skin care composition provided herein may be provided as lotion. Lotions offer ease of application, least irritation to the skin and optimal viscosity (e.g., in a range of about 5,000 to about 100,000 cps, about 10,000 to about 100,000 cps, about 20,000 to about 100,000 cps, about 30,000 to about 100,000 cps, about 40,000 to about 100,000 cps, about 50,000 to about 100,000 cps, about 60,000 to about 100,000 cps or about 70,000 to about 100,000 cps) that is needed for soothing and moisturizing effects on the epidermis of the skin, thereby providing beneficial effects on the impacted tissue or the tissue of interest for amelioration of skin disease conditions.

In certain exemplary, non-limiting embodiments, topical skin care composition may include ingredients in a specific amount for treating subjects with contact eczema/dermatitis, atopic eczema/dermatitis, seborrheic eczema/dermatitis, stasis eczema/dermatitis, nummular eczema/dermatitis, lichen simplex chronicus, dyshidrotic eczema/dermatitis, erythema, irritation, pruritus, dried skin, cracked skin, wrinkles and rosacea.

Ingredients may be provided in topical skin care composition provided herein, so long as they are physiologically acceptable and suitable for use in combination.

In certain exemplary, non-limiting embodiments, a topical skin care composition provided herein may include one or more skin conditioning agent(s), which may be a colloidal preparation of a grain product selected from the group consisting of oatmeal, cornmeal, soybean meal, rice meal, barley meal or other grains, combinations, and mixtures thereof.

In certain exemplary, non-limiting embodiments, a topical skin care composition provided herein may include about 0.01% to about 5% (w/w) of skin conditioning agent(s). For example, a topical skin care composition provided herein may comprise about 0.01% to about 5%, about 0.1% to about 5%, about 0.5% to about 5%, about 1% to about 5%, about 2% to about 5%, about 3% to about 5%, about 4% to about 5%, about 0.01% to about 4%, about 0.1% to about 4%, about 0.5% to about 4%, about 1% to about 4%, about 2% to about 4%, about 3% to about 4%, about 0.01% to about 3%, about 0.1% to about 3%, about 0.5% to about 3%, about 1% to about 3%, about 2% to about 3%, about 0.01% to about 2%, about 0.1% to about 2%, about 0.5% to about 2%, about 1% to about 2%, about 0.01% to about 1%, about 0.1% to about 1% or about 0.5% to about 1% (w/w) of skin conditioning agent(s).

In certain exemplary, non-limiting embodiments, a topical skin care composition provided herein may comprise at least 0.01%, at least 0.1%, at least 0.5%, at least 1%, at least 2%, at least 3%, at least 4% or at least 5% (w/w) of skin conditioning agent(s).

In certain exemplary, non-limiting embodiments, a topical skin care composition provided herein may comprise about 0.01%, about 0.1%, about 0.5%, about 1%, about 2%, about 3%, about 4% or about 5% (w/w) of skin conditioning agent(s).

In certain exemplary, non-limiting embodiments, a topical skin care composition provided herein may include one or more skin soothing agent(s), which may be a plant-derived skin care extract selected from Avena sativa (oat) kernel extract, Daucus carona sativa (carrot) extract, Arnica montana flower extract, Solanum lycopersicum (tomato) fruit/leaf/stem extract, Vaccinium angustifolium (blueberry) fruit extract, Aloe barbadensis leaf extract, Punica granatum (pomegranate) extract, Salix alba (willow) bark extract, Citrus aurantium dulcis (orange) fruit extract, Citrus medica limonum (lemon) fruit extract, Pyrus malus (apple) fruit extract, Vitis vinifera (grape) seed extract, Carica papaya (papaya) fruit extract, Lonicera caprifolium (honeysuckle) flower extract, Lonicera japonica (honeysuckle) flower extract, Fucus vesiculosus (seaweed) extract, Carica papaya (papaya) extract, Coffee arabica (coffee) seed extract, Glyzyrrhiza glabra (licorice) root extract, Citrus nobilis (mandarin orange) fruit extract, Citrullus vulgaris (watermelon) fruit extract, Euterpe oleracea (Acai) fruit extract, Silybum marianum fruit extract, combinations, and mixtures thereof.

In certain exemplary, non-limiting embodiments, a topical skin care composition provided herein may include about 1% to about 5% (w/w) of skin soothing agent(s). For example, a topical skin care composition provided herein may comprise about 1% to about 5%, about 2% to about 5%, about 3% to about 5%, about 4% to about 5%, about 1% to about 4%, about 2% to about 4%, about 3% to about 4%, about 1% to about 3%, about 2% to about 3% or about 1% to about 2% (w/w) of skin soothing agent(s).

In certain exemplary, non-limiting embodiments, a topical skin care composition provided herein may comprise at least 1%, at least 2%, at least 3%, at least 4% or at least 5% (w/w) of skin soothing agent(s).

In certain exemplary, non-limiting embodiments, a topical skin care composition provided herein may comprise about 1%, about 2%, about 3%, about 4% or about 5% (w/w) of skin soothing agent(s).

In certain exemplary, non-limiting embodiments, a topical skin care composition provided herein may include one or more occlusive(s), which may be a vegetable oil. Examples of vegetable oils include avocado oil, canola oil, coconut oil, corn oil, cotton seed oil, flax seed oil, grape seed oil, hemp seed oil, olive oil, palm oil, peanut oil, safflower oil, soybean oil, combinations, and mixtures thereof.

In certain exemplary, non-limiting embodiments, a topical skin care composition provided herein may include about 0.01% to about 5% (w/w) of occlusive(s). For example, a topical skin care composition provided herein may comprise about 0.01% to about 5%, about 0.1% to about 5%, about 0.5% to about 5%, about 1% to about 5%, about 2% to about 5%, about 3% to about 5%, about 4% to about 5%, about 0.01% to about 4%, about 0.1% to about 4%, about 0.5% to about 4%, about 1% to about 4%, about 2% to about 4%, about 3% to about 4%, about 0.01% to about 3%, about 0.1% to about 3%, about 0.5% to about 3%, about 1% to about 3%, about 2% to about 3%, about 0.01% to about 2%, about 0.1% to about 2%, about 0.5% to about 2%, about 1% to about 2%, about 0.01% to about 1%, about 0.1% to about 1% or about 0.5% to about 1% (w/w) of occlusive(s).

In certain exemplary, non-limiting embodiments, a topical skin care composition provided herein may comprise at least 0.01%, at least 0.1%, at least 0.5%, at least 1%, at least 2%, at least 3%, at least 4% or at least 5% (w/w) of occlusive(s).

In certain exemplary, non-limiting embodiments, a topical skin care composition provided herein may comprise about 0.01%, about 0.1%, about 0.5%, about 1%, about 2%, about 3%, about 4% or about 5% (w/w) of occlusive(s).

In certain exemplary, non-limiting embodiments, a topical skin care composition provided herein may include one or more emulsifier(s) which may be a polyglycerin, including polyglyceryl-10 dipalmitate, polyglyceryl-10 distearate, polyglyceryl-10 stearate, polyglyceryl-10 oleate; a sugar ester, including sorbitan palmitate, sorbitan stearate, sorbitan isostearate, sorbitan sesquistearate, sorbitan oleate, sorbitan sesquioleate, sorbitan olivate, sucrose cocoate; a glyceryl ester, including glyceryl stearate, glyceryl oleate; a hydrogenated emulsifier, including hydrogenated lecithin, hydrogenated palm glyceride, combinations, and mixtures thereof.

In certain exemplary, non-limiting embodiments, a topical skin care composition provided herein may include about 1% to about 10% (w/w) of emulsifier(s). For example, a topical skin care composition provided herein may comprise about 1% to about 10%, about 2% to about 10%, about 3% to about 10%, about 4% to about 10%, about 5% to about 10%, about 6% to about 10%, about 7% to about 10%, about 8% to about 10%, about 9% to about 10%, about 1% to about 9%, about 2% to about 9%, about 3% to about 9%, about 4% to about 9%, about 5% to about 9%, about 6% to about 9%, about 7% to about 9%, about 8% to about 9%, about 1% to about 8%, about 2% to about 8%, about 3% to about 8%, about 4% to about 8%, about 5% to about 8%, about 6% to about 8%, about 7% to about 8%, about 1% to about 7%, about 2% to about 7%, about 3% to about 7%, about 4% to about 7%, about 5% to about 7%, about 6% to about 7%, about 1% to about 6%, about 2% to about 6%, about 3% to about 6%, about 4% to about 6%, about 5% to about 6%, about 1% to about 5%, about 2% to about 5%, about 3% to about 5%, about 4% to about 5%, about 1% to about 4%, about 2% to about 4%, about 3% to about 4%, about 1% to about 3%, about 2% to about 3% or about 1% to about 2% (w/w) of emulsifier(s).

In certain exemplary, non-limiting embodiments, a topical skin care composition provided herein may comprise at least 1%, at least 2%, at least 3%, at least 4%, at least 5% (w/w), at least 6% (w/w), at least 7% (w/w), at least 8% (w/w), at least 9% (w/w) or at least 10% (w/w) of emulsifier(s).

In certain exemplary, non-limiting embodiments, a topical skin care composition provided herein may comprise about 1%, about 2%, about 3%, about 4%, about 5%, about 6%, about 7%, about 8%, about 9% or about 10% (w/w) of emulsifier(s).

In certain exemplary, non-limiting embodiments, a topical skin care composition provided herein may include first surfactant which may be a fatty alcohol selected from stearyl alcohol, cetostearyl alcohol, behenyl alcohol, myristyl alcohol, combinations, and mixtures thereof.

In certain exemplary, non-limiting embodiments, a topical skin care composition provided herein may include about 1% to about 10% (w/w) of first surfactant.

In certain exemplary, non-limiting embodiments, a topical skin care composition provided herein may comprise about 1%, about 2%, about 3%, about 4%, about 5%, about 6%, about 7%, about 8%, about 9% or about 10% (w/w) of first surfactant.

In certain exemplary, non-limiting embodiments, a topical skin care composition provided herein may include second surfactant which may be a non-ionic surfactant. Examples of non-ionic surfactants include polysorbate 20 (Tween 20), polysorbate 40 (Tween 40), polysorbate 60 (Tween 60), polysorbate 80 (Tween 80), combinations, and mixtures thereof.

In certain exemplary, non-limiting embodiments, a topical skin care composition provided herein may include about 1% to about 5% (w/w) of second surfactant.

In certain exemplary, non-limiting embodiments, a topical skin care composition provided herein may comprise about 1%, about 2%, about 3%, about 4% or about 5% (w/w) of second surfactant.

In certain exemplary, non-limiting embodiments, a topical skin care composition provided herein may include first emollient selected from group consisting of caprylic/capric triglyerides, castor oil, ceteareth-20, ceteareth-30, cetearyl alcohol, ceteth 20, cetostearyl alcohol, cetyl alcohol, cetyl stearyl alcohol, cocoa butter, diisopropyl adipate, glycerin, glyceryl monooleate, glyceryl monostearate, glyceryl stearate, isopropyl myristate, isopropyl palmitate, lanolin, lanolin alcohol, hydrogenated lanolin, liquid paraffins, linoleic acid, mineral oil, oleic acid, white petrolatum, polyethylene glycol, polyoxyethylene glycol fatty alcohol ethers, polyoxypropylene 15-stearyl ether, propylene glycol stearate, squalane, steareth-2 or -100, stearic acid, stearyl alcohol, combinations, and mixtures thereof.

In certain exemplary, non-limiting embodiments, a topical skin care composition provided herein may include about 1% to about 15% (w/w) of first emollient.

In certain exemplary, non-limiting embodiments, a topical skin care composition provided herein may comprise about 1%, about 2%, about 3%, about 4%, about 5%, about 6%, about 7%, about 8%, about 9%, about 10%, about 11%, about 12%, about 13%, about 14% or about 15% (w/w) of first emollient.

In certain exemplary, non-limiting embodiments, a topical skin care composition provided herein may include second emollient selected from group consisting of dimethicone, stearoxydimethicone or diisopropyl dimerate.

In certain exemplary, non-limiting embodiments, a topical skin care composition provided herein may include about 0.01% to about 5% (w/w) of second emollient.

In certain exemplary, non-limiting embodiments, a topical skin care composition provided herein may comprise about 0.01%, about 0.05%, about 0.1%, about 1%, about 2%, about 3%, about 4% or about 5% (w/w) of second emollient.

In certain exemplary, non-limiting embodiments, a topical skin care composition provided herein may include one or more humectant(s) which may be a polyol. Examples of humectants include glycerin, propylene glycol, butylene glycol, dipropylene glycol, pentylene glycol, hexylene glycol, polyethylene glycol, combinations, and mixtures thereof.

In certain exemplary, non-limiting embodiments, a topical skin care composition provided herein may include about 0.01% to about 5% (w/w) of humectant(s).

In certain exemplary, non-limiting embodiments, a topical skin care composition provided herein may comprise about 0.01%, about 0.05%, about 0.1%, about 1%, about 2%, about 3%, about 4% or about 5% (w/w) of humectant(s).

In certain exemplary, non-limiting embodiments, a topical skin care composition provided herein may include one or more penetration enhancer(s) which may be a glycol ether, unsaturated fatty acid or alcohol. Examples include ethoxydiglycol, steareth-20, steareth-2, octyl decanol, isopropyl myristate, oleic acid, propylene glycol, combinations, and mixtures thereof.

In certain exemplary, non-limiting embodiments, a topical skin care composition provided herein may include about 1% to about 10% (w/w) of penetration enhancer(s).

In certain exemplary, non-limiting embodiments, a topical skin care composition provided herein may comprise about 1%, about 2%, about 3%, about 4%, about 5%, about 6%, about 7%, about 8%, about 9% or about 10% (w/w) of penetration enhancer(s).

In certain exemplary, non-limiting embodiments, a topical skin care composition provided herein may include one or more opacifying agent(s) selected from the group consisting of silica, titanium (IV) oxide, zinc oxide, mica, combinations, and mixtures thereof.

In certain exemplary, non-limiting embodiments, a topical skin care composition provided herein may include about 0.01% to about 3% (w/w) of opacifying agent(s).

In certain exemplary, non-limiting embodiments, a topical skin care composition provided herein may comprise about 0.01%, about 0.05%, about 0.1%, about 0.5%, about 1%, about 1.5%, about 2%, about 2.5% or about 3% (w/w) of opacifying agent(s).

In certain exemplary, non-limiting embodiments, a topical skin care composition provided herein may include one or more stabilizer(s) which may be an acid, base, complexing agent or a mixture thereof, preferably selected from the group consisting of acetylacetonate, crown ethers, cryptands, ethylenediaminetriacetate and its salts, ethylenediaminetetraacetate (EDTA) and its salts, nitrilotriacetate (NTA) and its salts, citric acid and its salts, triethylentetramine, porphin, poly acrylate, poly asparagate, acidic peptides, phthalocyanin, salicylate, glycinate, lactate, propylendiamine, ascorbate, oxalic acid and its salts, acetic acid and its salts, ammonia and its salts, combinations, and mixtures thereof.

In certain exemplary, non-limiting embodiments, a topical skin care composition provided herein may include about 0.01% to about 5% (w/w) of stabilizer(s).

In certain exemplary, non-limiting embodiments, a topical skin care composition provided herein may comprise about 0.01%, about 0.1%, about 1%, about 2%, about 3%, about 4% or about 5% (w/w) of stabilizer(s).

In certain exemplary, non-limiting embodiments, a topical skin care composition provided herein may include one or more preservative(s), such as ethyl paraben, methylparaben, propylparaben, butylparaben, isobutyl paraben, benzalkonium chloride, imidurea, phenoxyethanol SA, ethylenediaminetetraacetic acid (EDTA), combinations, and mixtures thereof.

In certain exemplary, non-limiting embodiments, a topical skin care composition provided herein may include about 0.1% to about 3% (w/w) of preservative(s).

In certain exemplary, non-limiting embodiments, a topical skin care composition provided herein may comprise about 0.1%, about 0.5%, about 1%, about 2%, or about 3% (w/w) of preservative(s).

In certain exemplary, non-limiting embodiments, a topical skin care composition provided herein may include one or more pH adjusting agent(s)/buffering agent(s), such as calcium hydroxide, sodium hydroxide, potassium hydroxide, trisodium citrate dihydrate, citric acid monohydrate, acetate/acetic acid, phosphate/phosphoric acid, formate/formic acid, propionate/propionic acid, lactate/lactic acid, carbonate/carbonic acid, ammonium/ammonia buffer, triethylamine, triethanolamine, combinations, and mixtures thereof. In certain exemplary, non-limiting embodiments, the composition may have a pH within the range of about 5 to about 7.

In certain exemplary, non-limiting embodiments, a topical skin care composition provided herein may include one or more carriers, such as water, alcohol, mineral oil, combinations, and mixtures thereof.

In certain exemplary, non-limiting embodiments, a topical skin care composition provided herein may retain a viscosity in a range of about 5,000 to about 100,000 cps and a pH within a range of about 5 to about 7 while being cycled weekly between freezer and ambient room temperature conditions for a minimum of 1 month.

It is understood that the amount of ingredients necessary to achieve a desired skin care result is influenced by, and will therefore vary based on, a number of factors, including for example and without limitation, the age, sex, and weight of the subject, factors that influence the metabolic rate, and the specific disorders, diseases or related treatment symptoms of the subject.

One of skill in the art will understand that the ingredients in the final formulations must total 100% and, based on the teachings provided herein, will understand that modifications to the exemplary formulations provided herein are possible (e.g., replacement of a recited ingredient with a different ingredient, addition of a different ingredient, and/or modification of an amount of an ingredient) provided that such modifications result in a formulation as taught and described herein.

In another aspect, there is provided a method of applying a topical skin care composition provided herein to the skin of a subject. In some embodiments, the skin is affected by a skin disease or condition. Application may be carried out by dropping, spraying, diffusing, dispersing, squirting, or spreading the composition, and may optionally be carried out using an applicator, such as a dropper, a nebulizer, an impregnated gauze sheet, a syringe, or a cotton swab.

In another aspect, there is provided a method of treating a skin disease or condition in a subject, comprising applying a topical composition provided herein to the skin of a subject.

In another aspect, there is provided a use of a topical skin care composition provided herein for the treatment of a skin disease or condition in a subject.

In certain exemplary, non-limiting embodiments, the skin disease or condition is: contact eczema/dermatitis, atopic eczema/dermatitis, seborrheic eczema/dermatitis, stasis eczema/dermatitis, nummular eczema/dermatitis, lichen simplex chronicus, dyshidrotic eczema/dermatitis, erythema, irritation, pruritus, dried skin, cracked skin, wrinkles and rosacea.

Still further embodiments of the present disclosure relate to providing a kit, which may include a container containing a topical skin care composition provided herein, or a number of containers containing materials for preparing the topical skin care composition. The kit may also include instructions for treating a skin disease or condition using the topical skin care composition including dosage and how the composition may be applied to the skin. When separate containers are provided in the kit, and depending on the contents in these containers, the kit may also include instructions for preparing a topical skin care composition, or compositions with different concentrations of ingredients, from the materials included in the kit and optionally other materials such as a carrier or other additives. The kit may further include an applicator for applying the topical skin care composition to the skin of a subject and may include specific instructions on how to use the applicator.

In certain exemplary, non-limiting embodiments, a topical skin care composition provided herein may be prepared or obtained from a kit comprising (a) one or more cannabinoids; (b) ingredients; and (c) instructions in the kit, and wherein the instructions comprise information allowing all of (b) be mixed with (a) at selected concentrations disclosed herein. The kit may include separate containers or instructions for providing or preparing more than one composition with different concentrations for one or more of (a) and (b).

In certain exemplary, non-limiting embodiments, a kit may include a container containing a topical skin care composition provided herein. The composition may be in form of cream, ointment, gel, lotion, liquid or the like as described above. The container may be, for example, a liquid bottle or a paste tube depending on the physical form of the composition. In other embodiments, a kit may include a plurality of containers containing materials for forming a topical skin care composition provided herein. The kit may further comprise at least one of instructions for applying the composition to skin; instructions for using the composition to treat a skin disease or condition according to the methods or uses provided herein; and instructions for using the materials in the plurality of containers to prepare the composition according to the methods of preparation provided herein. Optional components of a kit may include one or more applicators (such as droppers, sprayers, gauze sheets, and cotton-tipped applicators) for applying the composition to skin. The one or more applicators may be sterilized and contained in a sealed sterile packaging.

The discussion herein and the following examples set forth and illustrate various exemplary embodiments of the present disclosure, which are understood to be illustrative and non-limiting.

### Non-Limiting Embodiments

Particular embodiments of the disclosure include, without limitation, the following:
1. A topical skin care composition comprising:
   a. a cannabinoid at 0.01-10% (w/w),
   b. a skin conditioning agent at 0.01-5% (w/w),
   c. a skin soothing agent at 1-5% (w/w),
   d. an occlusive at 0.01-5% (w/w),
   e. an emulsifier at 1-10% (w/w),
   f. water to make up 100% by weight.
   wherein the topical skin care composition comprises less than 20% (w/w) of fatty alcohol.
2. The topical skin care composition of embodiment 1, wherein the topical skin care composition comprises less than 10% (w/w) of fatty alcohol.
3. The topical skin care composition of embodiment 1 or 2, wherein the topical skin care composition comprises less than 5% (w/w) of fatty alcohol.
4. A topical skin care composition comprising:
   a. a cannabinoid at 0.01-10% (w/w),
   b. a skin conditioning agent at 0.01-5% (w/w),
   c. a skin soothing agent at 1-5% (w/w),
   d. an occlusive at 0.01-5% (w/w),
   e. an emulsifier at 1-10% (w/w),
   f. water no less than 51% (w/w).
5. The topical skin care composition of any one of embodiments 1 to 4, which comprises about 0.1% (w/w) of the cannabinoid.
6. The topical skin care composition of any one of embodiments 1 to 4, which comprises about 0.5% (w/w) of the cannabinoid.
7. The topical skin care composition of any one of embodiments 1 to 4, which comprises about 1% (w/w) of the cannabinoid.
8. The topical skin care composition of any one of embodiments 1 to 4, which comprises about 2% (w/w) of the cannabinoid.
9. The topical skin care composition of any one of embodiments 1 to 4, which comprises about 3% (w/w) of the cannabinoid.
10. The topical skin care composition of any one of embodiments 1 to 4, which comprises about 4% (w/w) of the cannabinoid.
11. The topical skin care composition of any one of embodiments 1 to 4, which comprises about 5% (w/w) of the cannabinoid.
12. The topical skin care composition of any one of embodiments 1 to 4, which comprises about 6% (w/w) of the cannabinoid.
13. The topical skin care composition of any one of embodiments 1 to 4, which comprises about 7% (w/w) of the cannabinoid.
14. The topical skin care composition of any one of embodiments 1 to 4, which comprises about 8% (w/w) of the cannabinoid.
15. The topical skin care composition of any one of embodiments 1 to 4, which comprises about 9% (w/w) of the cannabinoid.
16. The topical skin care composition of any one of embodiments 1 to 4, which comprises about 10% (w/w) of the cannabinoid.
17. The topical skin care composition of any one of embodiments 1 to 16, wherein the cannabinoid is phytocannabinoid.
18. The topical skin care composition of any one of embodiments 1 to 17, wherein the phytocannabinoid is tetrahydrocannabinol (THC), tetrahydrocannabinolic acid (THCA), cannabidiol (CBD), cannabidiolic acid (CBDA), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigervarin (CBGV), cannabigerol monomethyl ether (CBGM), cannabielsoin (CBE), cannabicitran (CBT), or any combination thereof.
19. The topical skin care composition of embodiment 18, wherein the cannabinoid is cannabidiol (CBD).
20. The topical skin care composition of embodiments 1 to 19, which comprises about 0.01% (w/w) of skin conditioning agent.
21. The topical skin care composition of embodiments 1 to 19, which comprises about 0.1% (w/w) of skin conditioning agent.
22. The topical skin care composition of embodiments 1 to 19, which comprises about 0.5% (w/w) of skin conditioning agent.
23. The topical skin care composition of embodiments 1 to 19, which comprises about 1% (w/w) of skin conditioning agent.
24. The topical skin care composition of embodiments 1 to 19, which comprises about 2% (w/w) of skin conditioning agent.
25. The topical skin care composition of embodiments 1 to 19, which comprises about 3% (w/w) of skin conditioning agent.
26. The topical skin care composition of embodiments 1 to 19, which comprises about 4% (w/w) of skin conditioning agent.
27. The topical skin care composition of embodiments 1 to 19, which comprises about 5% (w/w) of skin conditioning agent.
28. The topical skin care composition of embodiments 1 to 27, wherein the skin conditioning agent is a colloidal preparation of oatmeal, cornmeal, soybean meal, rice meal, barley meal or other grains, or any combination thereof.
29. The topical skin care composition of embodiment 28, wherein the skin conditioning agent is colloidal preparation of oatmeal.
30. The topical skin care composition of embodiments 1 to 29, which comprises about 1% (w/w) of skin soothing agent.
31. The topical skin care composition of embodiments 1 to 29, which comprises about 2% (w/w) of skin soothing agent.
32. The topical skin care composition of embodiments 1 to 29, which comprises about 3% (w/w) of skin soothing agent.
33. The topical skin care composition of embodiments 1 to 29, which comprises about 4% (w/w) of skin soothing agent.
34. The topical skin care composition of embodiments 1 to 29, which comprises about 5% (w/w) of skin soothing agent.
35. The topical skin care composition of embodiments 1 to 34, wherein the skin soothing agent is a plant-derived extract selected from Avena sativa (oat) kernel extract, Daucus carona sativa (carrot) extract, Arnica montana flower extract, Solanum lycopersicum (tomato) fruit/leaf/stem extract, Vaccinium angustifolium (blueberry) fruit extract, Aloe barbadensis leaf extract, Punica granatum (pomegranate) extract, Salix alba (willow) bark extract, Citrus aurantium dulcis (orange) fruit extract, Citrus medica limonum (lemon) fruit extract, Pyrus malus (apple) fruit extract, Vitis vinifera (grape) seed extract, Carica papaya (papaya) fruit extract, Lonicera caprifolium (honeysuckle) flower extract, Lonicera japonica (honeysuckle) flower extract, Fucus vesiculosus (seaweed) extract, Carica papaya (papaya) extract, Coffee arabica (coffee) seed extract, Glyzyrrhiza glabra (licorice) root extract, Citrus nobilis (mandarin orange) fruit extract, Citrullus vulgaris (watermelon) fruit extract, Euterpe oleracea (Acai) fruit extract, Silybum marianum fruit extract, or any combination thereof.
36. The topical skin care composition of embodiment 35, wherein the skin soothing agent is Avena sativa (oat) kernel extract.
37. The topical skin care composition of embodiments 1 to 36, which comprises about 0.01% (w/w) of occlusive.
38. The topical skin care composition of embodiments 1 to 36, which comprises about 0.1% (w/w) of occlusive.
39. The topical skin care composition of embodiments 1 to 36, which comprises about 0.5% (w/w) of occlusive.
40. The topical skin care composition of embodiments 1 to 36, which comprises about 1% (w/w) of occlusive.
41. The topical skin care composition of embodiments 1 to 36, which comprises about 2% (w/w) of occlusive.
42. The topical skin care composition of embodiments 1 to 36, which comprises about 3% (w/w) of occlusive.
43. The topical skin care composition of embodiments 1 to 36, which comprises about 4% (w/w) of occlusive.
44. The topical skin care composition of embodiments 1 to 36, which comprises about 5% (w/w) of occlusive.
45. The topical skin care composition of embodiments 1 to 44, wherein the occlusive is a vegetable oil selected from avocado oil, canola oil, coconut oil, corn oil, cotton seed oil, flax seed oil, grape seed oil, hemp seed oil, olive oil, palm oil, peanut oil, safflower oil, soybean oil, or any combination thereof.
46. The topical skin care composition of embodiment 45, wherein the occlusive is hemp seed oil.
47. The topical skin care composition of embodiments 1 to 46, which comprises about 1% (w/w) of emulsifier.
48. The topical skin care composition of embodiments 1 to 46, which comprises about 2% (w/w) of emulsifier.
49. The topical skin care composition of embodiments 1 to 46, which comprises about 3% (w/w) of emulsifier.
50. The topical skin care composition of embodiments 1 to 46, which comprises about 4% (w/w) of emulsifier.
51. The topical skin care composition of embodiments 1 to 46, which comprises about 5% (w/w) of emulsifier.
52. The topical skin care composition of embodiments 1 to 46, which comprises about 6% (w/w) of emulsifier.
53. The topical skin care composition of embodiments 1 to 46, which comprises about 7% (w/w) of emulsifier.
54. The topical skin care composition of embodiments 1 to 46, which comprises about 8% (w/w) of emulsifier.
55. The topical skin care composition of embodiments 1 to 46, which comprises about 9% (w/w) of emulsifier.
56. The topical skin care composition of embodiments 1 to 46, which comprises about 10% (w/w) of emulsifier.
57. The topical skin care composition of embodiments 1 to 56, wherein the emulsifier is a polyglycerin selected from polyglyceryl-10 dipalmitate, polyglyceryl-10 distearate, polyglyceryl-10 stearate, polyglyceryl-10 oleate; a sugar ester, including sorbitan palmitate, sorbitan stearate, sorbitan isostearate, sorbitan sesquistearate, sorbitan oleate, sorbitan sesquioleate, sorbitan olivate, sucrose cocoate; a glyceryl ester, including glyceryl stearate, glyceryl oleate; a hydrogenated emulsifier, including hydrogenated lecithin, hydrogenated palm glyceride, or any combination thereof.
58. The topical skin care composition of embodiment 57, wherein the emulsifier is glyceryl stearate.
59. The topical skin care composition of embodiments 1 to 58, wherein the composition further comprises a first surfactant at 1-10% (w/w).
60. The topical skin care composition of embodiment 59, wherein the first surfactant is stearyl alcohol, cetostearyl alcohol, behenyl alcohol, myristyl alcohol, or any combination thereof.
61. The topical skin care composition of embodiment 60, wherein the first surfactant is cetostearyl alcohol.
62. The topical skin care composition of embodiments 1 to 61, wherein the composition further comprises a second surfactant at 1-5% (w/w).
63. The topical skin care composition of embodiment 62, wherein the second surfactant is polysorbate 20 (Tween 20), polysorbate 40 (Tween 40), polysorbate 60 (Tween 60), polysorbate 80 (Tween 80), or any combination thereof.
64. The topical skin care composition of embodiment 63, wherein the second surfactant is polysorbate 60 (Tween 60).
65. The topical skin care composition of embodiments 1 to 64, wherein the composition further comprises a first emollient at 1-15% (w/w).
66. The topical skin care composition of embodiment 65, wherein the first emollient is caprylic/capric triglyerides, castor oil, ceteareth-20, ceteareth-30, cetearyl alcohol, ceteth 20, cetostearyl alcohol, cetyl alcohol, cetyl stearyl alcohol, cocoa butter, diisopropyl adipate, glycerin, glyceryl monooleate, glyceryl monostearate, glyceryl stearate, isopropyl myristate, isopropyl palmitate, lanolin, lanolin alcohol, hydrogenated lanolin, liquid paraffins, linoleic acid, mineral oil, oleic acid, white petrolatum, polyethylene glycol, polyoxyethylene glycol fatty alcohol ethers, polyoxypropylene 15-stearyl ether, propylene glycol stearate, squalane, steareth-2 or -100, stearic acid, stearyl alcohol, or any combination thereof.
67. The topical skin care composition of embodiment 66, wherein the first emollient is white petrolatum.
68. The topical skin care composition of embodiments 1 to 67, wherein the composition further comprises a second emollient at 0.01-5% (w/w).
69. The topical skin care composition of embodiment 68, wherein the second emollient is dimethicone, stearoxydimethicone, diisopropyl dimerate, or any combination thereof.
70. The topical skin care composition of embodiment 69, wherein the second emollient is dimethicone.
71. The topical skin care composition of embodiments 1 to 70, wherein the composition further comprises a humectant at 0.01-5% (w/w).
72. The topical skin care composition of embodiment 71, wherein the humectant is glycerin, propylene glycol, butylene glycol, dipropylene glycol, pentylene glycol, hexylene glycol, polyethylene glycol, or any combination thereof.
73. The topical skin care composition of embodiment 72, wherein the humectant is glycerin.
74. The topical skin care composition of embodiments 1 to 73, wherein the composition further comprises a penetration enhancer at 1-10% (w/w).
75. The topical skin care composition of embodiment 74, wherein the penetration enhancer is ethoxydiglycol, steareth-20, steareth-2, octyl decanol, isopropyl myristate, oleic acid, propylene glycol, or any combination thereof.
76. The topical skin care composition of embodiment 75, wherein the penetration enhancer is ethoxydiglycol.
77. The topical skin care composition of embodiments 1 to 76, wherein the composition further comprises an opacifying agent at 0.01-3% (w/w).
78. The topical skin care composition of embodiment 77, wherein the opacifying agent is silica, titanium (IV) oxide, zinc oxide, mica, or any combination thereof.
79. The topical skin care composition of embodiment 78, wherein the opacifying agent is titanium (IV) oxide.
80. The topical skin care composition of embodiments 1 to 79, wherein the composition further comprises a stabilizer at 0.01-5% (w/w).
81. The topical skin care composition of embodiment 80, wherein the stabilizer is acetylacetonate, crown ethers, cryptands, ethylenediaminetriacetate and its salts, ethylenediaminetetraacetate (EDTA) and its salts, nitrilotriacetate (NTA) and its salts, citric acid and its salts, triethylentetramine, porphin, poly acrylate, poly asparagate, acidic peptides, phthalocyanin, salicylate, glycinate, lactate, propylendiamine, ascorbate, oxalic acid and its salts, acetic acid and its salts, ammonia and its salts, or any combination thereof.
82. The topical skin care composition of embodiment 81, wherein the stabilizer is disodium salt of ethylenediaminetetraacetic acid (EDTA).
83. The topical skin care composition of embodiments 1 to 82, wherein the composition further comprises a preservative at 0.01-3% (w/w).
84. The topical skin care composition of embodiment 83, wherein the preservative is ethyl paraben, methylparaben, propylparaben, butylparaben, isobutyl paraben, benzalkonium chloride, imidurea, phenoxyethanol SA, ethylenediaminetetraacetic acid (EDTA), or any combination thereof.
85. The topical skin care composition of embodiment 84, wherein the preservative is phenoxyethanol SA.
86. The topical skin care composition of embodiments 1 to 85, wherein the composition further comprises a pH adjusting agent/buffering agent.
87. The topical skin care composition of embodiment 86, wherein the pH adjusting agent/buffering agent is calcium hydroxide, sodium hydroxide, potassium hydroxide, trisodium citrate dihydrate, citric acid monohydrate, acetate/acetic acid, phosphate/phosphoric acid, formate/formic acid, propionate/propionic acid, lactate/lactic acid, carbonate/carbonic acid, ammonium/ammonia buffer, triethylamine, triethanolamine, or any combination thereof.
88. The topical skin care composition of embodiment 87, wherein the pH adjusting agent/buffering agent is a combination of trisodium citrate dihydrate and citric acid monohydrate.
89. The topical skin care composition of any one of embodiments 1 to 88, wherein the composition comprises one or more carriers, such as water, alcohol, mineral oil, or any combination thereof.
90. The topical skin care composition of any one of embodiments 1 to 89, wherein the composition has a pH within the range of about 5 to about 7.
91. The topical skin care composition of any one of embodiments 1 to 90, wherein the composition has a viscosity in a range of about 5,000 to about 100,000 cps.
92. The topical skin care composition of any one of embodiments 1 to 91, wherein the composition has a viscosity in a range of about 5,000 to about 100,000 cps and a pH within a range of about 5 to about 7, and retains its viscosity and pH within the said ranges while being cycled weekly between freezer and ambient room temperature conditions for a minimum of 3 months.
93. The topical skin care composition of any one of embodiments 1 to 92, wherein the composition is a cream, ointment, gel, lotion, liquid, solution, spray, aerosol, any other dosage forms suitable for topical application, or any combination thereof.
94. The topical skin care composition of embodiment 93, wherein the composition is a lotion.
95. A method comprising applying the topical skin care composition of any one of embodiments 1 to 94 to the skin of a subject.
96. The method of embodiment 95, wherein the skin of the subject is affected by a skin disease or condition.
97. The method of embodiment 96, wherein the skin disease or condition is: contact eczema/dermatitis, atopic eczema/dermatitis, seborrheic eczema/dermatitis, stasis eczema/dermatitis, nummular eczema/dermatitis, lichen simplex chronicus, dyshidrotic eczema/dermatitis, erythema, irritation, pruritus, dried skin, cracked skin, wrinkles and rosacea.
98. A method of treating a skin disease or condition in a subject, comprising applying the topical skin care composition of any one of embodiments 1 to 97 to the skin of a subject.
99. The method of embodiment 98, wherein the skin disease or condition is: contact eczema/dermatitis, atopic eczema/dermatitis, seborrheic eczema/dermatitis, stasis eczema/dermatitis, nummular eczema/dermatitis, lichen simplex chronicus, dyshidrotic eczema/dermatitis, erythema, irritation, pruritus, dried skin, cracked skin, wrinkles and rosacea.
100. The method of any one of embodiments 95 to 99, wherein the applying comprises dropping, spraying, diffusing, dispersing, squirting, or spreading the composition.
101. The method of any one of embodiments 95 to 100, wherein the subject is a mammal.
102. The method of any one of embodiments 95 to 101, wherein the subject is a companion animal.
103. The method of embodiment 101, wherein the subject is a human.
104. The method of any one of embodiments 95 to 103, wherein the method further comprises administering an additional therapy for a skin disease or condition.
105. Use of the topical skin care formulation of any one of embodiments 1 to 94 for the treatment of a skin disease or condition in a subject.
106. The use of embodiment 105, wherein the skin disease or condition is: contact eczema/dermatitis, atopic eczema/dermatitis, seborrheic eczema/dermatitis, stasis eczema/dermatitis, nummular eczema/dermatitis, lichen simplex chronicus, dyshidrotic eczema/dermatitis, erythema, irritation, pruritus, dried skin, cracked skin, wrinkles and rosacea.
107. The use of any one of embodiments 105 or 106, wherein the subject is a mammal.
108. The use of any one of embodiments 105 to 107, wherein the subject is a companion animal.
109. The use of embodiment 107, wherein the subject is a human.
110. The use of any one of embodiments 105 to 109, wherein the treatment further comprises an additional therapy for a skin disease or condition.
111. A kit comprising a container containing the topical skin care composition of any one of embodiments 1 to 94.
112. A kit comprising a plurality of containers containing materials for forming the topical skin care composition of any one of embodiments 1 to 94.
113. The kit of embodiment 111 or 112, further comprising instructions for preparing the topical skin care composition from the materials in the containers.
114. The kit of any one of embodiments 111 to 113, further comprising instructions for applying the topical skin care composition to the skin of a subject.
115. The kit of any one of embodiments 111 to 114, further comprising instructions for using the topical skin care composition to treat a skin disease or condition according to the method of any one of embodiments 95 to 104.
116. The kit of any one of embodiments 111 to 115, further comprising one or more applicators for applying the topical skin care composition to the skin of a subject.
117. A topical skin care composition comprising:
   a. a cannabinoid at 0.01-10% (w/w),
   b. a skin conditioning agent at 0.01-5% (w/w),
   c. a skin soothing agent at 1-5% (w/w),
   d. an occlusive at 0.01-5% (w/w),
   e. an emulsifier at 1-10% (w/w),
   f. a first surfactant at 1-10% (w/w),
   g. a second surfactant at 1-5% (w/w),
   h. a first emollient at 1-15% (w/w),
   i. a second emollient at 0.01-5% (w/w),
   j. a humectant at 0.01-5% (w/w),
   k. a penetration enhancer at 1-10% (w/w),
   l. an opacifying agent at 0.01-3% (w/w),
   m. a stabilizer at 0.01-5% (w/w),
   n. a preservative at 0.01-3% (w/w),
   o. pH adjusting agents in a quantity sufficient for the composition to maintain a pH of 5-7, and
   p. water to make up 100% by weight.
118. The topical skin care composition of embodiment 129, wherein said cannabinoid is CBD, skin conditioning agent is colloidal oatmeal, skin soothing agent is oat kernel extract, occlusive is hemp seed oil, emulsifier is glyceryl stearate, first surfactant is cetostearyl alcohol, second surfactant is Tween 60, first emollient is white petrolatum, second emollient is dimethicone, humectant is glycerin, penetration enhancer is ethoxydiglycol, opacifying agent is titanium (IV) oxide, stabilizer is disodium salt of ethylenediaminetetraacetic acid (EDTA), preservative is phenoxyethanol SA and pH adjusting agents are trisodium citrate dihydrate and citric acid monohydrate.
119. The topical skin care composition of embodiment 117, wherein the said composition has a pH within the range of about 5 to 7.
120. The topical skin care composition of embodiment 117, wherein the said composition has a viscosity in a range of about 5,000 to about 100,000 cps.
121. The topical skin care composition of embodiment 117, wherein the said composition is a topical lotion composition.
122. The topical skin care composition of any one of embodiments 117 to 121, wherein the composition is useful for the treatment of one or more skin diseases or conditions such as: contact eczema/dermatitis, atopic eczema/dermatitis, seborrheic eczema/dermatitis, stasis eczema/dermatitis, nummular eczema/dermatitis, lichen simplex chronicus, dyshidrotic eczema/dermatitis, erythema, irritation, pruritus, dried skin, cracked skin, wrinkles and rosacea.
123. A lotion comprising:
   a. a cannabinoid at 0.01-10% (w/w),
   b. a skin conditioning agent at 0.01-5% (w/w),
   c. an emulsifying agent 1-10% (w/w),
   d. an occlusive at 0.01-5% (w/w), and
   e. water to make up 100% by weight.
124. A lotion of embodiment 123, wherein the composition has a viscosity in a range of about 5,000 to about 100,000 cps.

### Non-Limiting Examples

The following examples are provided to illustrate certain particular features and/or embodiments. These examples should not be construed to limit the disclosure to the particular features or embodiments described.

Example 1. Topical skin care composition: This example provides an overview of embodiments of the skin care composition described herein, which is useful in the topical treatment of skin conditions such as eczema and/or can help reduce itch, inflammation, dryness and redness.

TABLE 1
Composition

| Phase | Ingredient | w/w % |
|---|---|---|
| Oil | Gelot ™ 64 | 3.00 |
| Phase | Transcutol P | 3.00 |
| (A) | Cetostearyl Alcohol | 5.00 |
| | White Petrolatum | 9.00 |
| | Glycerin | 1.50 |
| | Dimethicone | 1.50 |
| | Cannabidiol | 0.50 |
| Aqueous | Aqua | 65.55 |
| Phase | Trisodium Citrate Dihydrate | 1.235 |
| (B) | Citric Acid Monohydrate | 0.311 |
| | Titanium (IV) Oxide | 0.20 |
| | Titriplex III Disodium EDTA Dihydrate | 0.70 |
| | Tween 60 | 4.00 |
| | Colloidal Oatmeal | 1.00 |
| | Oat Kernel Extract | 2.00 |
| Additive | Hemp Seed Oil | 1.0 |
| Phase | Phenoxyethanol | 0.50 |
| (C) | | |

| | |
|---|---|
| Total | 100.00 |

Example 2. Method of manufacturing: The following procedure was used to produce a laboratory batch according to the formula in Table 1.

### I. Preparation of Phase A (Oil Phase)

a. Individually weigh out the following ingredients and dispense directly into Vessel A: Transcutol P, White petrolatum, Glycerin, Dimethicone.
b. In a weighing boat, measure Gelot 64, Cetostearyl Alcohol.
c. Add a magnetic stirrer and place the vessel on Heat/Stir Element.
d. Increase the temperature to 70-75° C. under continuous stirring. Mix well until homogenous.
e. Weigh and dispense Cannabidiol (CBD) using a weighing boat into Vessel A.

### II. Preparation of Phase B (Aqueous Phase)

a. Dispense water into Vessel B and heat to 65° C.
b. Add a magnetic stir bar to Vessel B and place the vessel on the Heat/Stir Element.
c. Weigh out the following ingredients and dispense into Vessel B: Trisodium Citrate, Dihydrate; Citric Acid, Anhydrous; Titanium (IV) Oxide; Titriplex III Disodium EDTA Dihydrate; Tween 60; Colloidal Oatmeal, Oat Kernel Extract.
d. Maintain the temperature at 65° C. under continuous stirring until ingredients are dissolved.

### III. Preparation of Phase C (Additive Phase)

a. Weigh out the following ingredients and dispense into Vessel C: Hemp Seed Oil, Phenoxyethanol.
b. Add a magnetic stir bar to Vessel C and place the vessel on the Heat/Stir Element.
c. Without applying heat, mix thoroughly under continuous stirring using the Heating/Stirring Element until everything is dissolved.

Example 3. Topical skin care composition for hydration and erythema of the skin: An open-label, randomized trial (ClinicalTrials.gov Identifier: NCT04045314).

This example describes a clinical study to evaluate the short (i.e., after single application) and long-term (i.e., after periodic application) hydrating effects of composition described in Example 1 on skin of the volar forearm and facial skin and to assess the effects on erythema, functional properties of the skin and irritation.

Outcomes

Primary outcomes were to evaluate the hydrating effects of single and periodic application of the composition on the skin of the volar forearm and face, through the direct measurement of its electrical properties as an indicator of water content at 1 and 3 hours (short-term effects) and at 2 and 4 weeks (long-term effects).

Secondary outcomes were to assess the short-term and long-term effects on instrumentally assessed erythema, viscoelastic properties of the skin, functional properties of the skin through the measurement of trans-epidermal water loss (TEWL) and appearance of evaluator assessed irritation by visual scoring. Additionally, sensation and tolerance after single and periodic applications were assessed using a questionnaire.

Inclusion Criteria

Healthy adult men or women with dry skin (Modified Kligman test >0) were included.

Exclusion Criteria

The following is full list of the exclusion criteria:

Pregnant or breast-feeding women.

Subjects with a chronic disease that requires medication.

Subjects with known diagnosis of cancer.

Smoking habit or alcohol consumption habit (i.e., once a day or more).

Recreational or medicinal use of cannabinoids.

Skin diseases (i.e., diseases that require care of a dermatologist).

Current medication uses such as: Immunomodulators, antibiotics, corticoids or retinoids.

Hypersensitivity to any component of the research product.

Involvement in other clinical or cosmetic studies in the last 6 months.

Recent exposure to sun causing sun tanning (i.e., as reported by the subject causing discomfort or change in the usual appearance of the skin).

Permanent decoration of the skin in the test area.

Methods

Healthy adult men and women aged 18 to 70 years with dry skin were enrolled (n=61) into a single group, randomized, passive-controlled, open label trial of composition containing 0.5% cannabidiol. The composition was administered topically to subjects and were evaluated for short term outcomes during their first visit. To evaluate the long-term effects, two other visits were made at intervals of 2 weeks.

The study design considered intra-individual control with contralateral arm; allocation of patients was sequential. Once baseline characteristics were established, control arm was randomized using a specific function of the data capture platform that ensures 1:1 of control arm for all patients.

All the information collected in the short-term period the data was analyzed in order to ensure completeness and coherence. For descriptive analysis, quantitative variables are summarized using measures of central tendency and dispersion (means, median, standard deviation, ranges). Qualitative variables, both nominal and ordinal, were described through absolute and relative frequencies.

To perform hypothesis testing, Student's T was used to evaluate the primary endpoint at the end of the short-term period, additionally, ANOVA was used to evaluate all other time points. However, as some instrumental measurements (i.e., viscoelasticity and TEWL) deviated significantly from normality, Kruskal-Wallis test was used to perform hypothesis testing followed by post-hoc Tukey test to assess pair-wise comparisons.

Results

61 subjects were assessed for eligibility during the screening and 49 subjects completed the short-term and long-term analysis. Subjects who met the selection criteria and failed to complete the study were dropped during the washout due to not compliance or unwillingness to comply with the washout conditions.

Effects on Skin Hydration

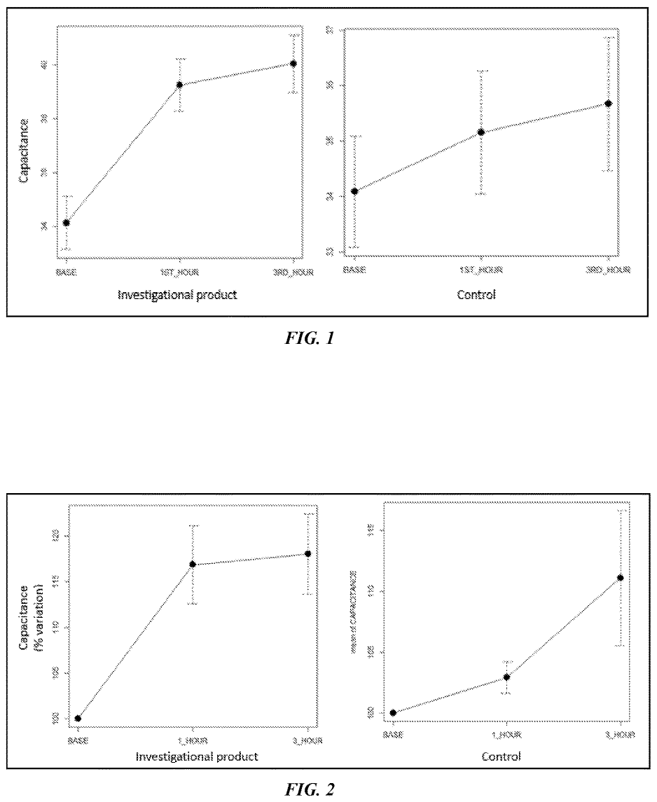

The hydrating effects of single application of the composition on the skin of the volar forearm and face was assessed through the direct measurement of its electrical properties (capacitance) as an indicator of water content at 1 and 3 hours. The following values of capacitance at baseline, as well as at 1 and 3 hours after application of composition versus control were obtained (Table 2 & FIG. 1).

TABLE 2
Mean capacitance and 95% confidence interval (CI)

| | | Investigational | |
|---|---|---|---|
| | | Product | Control |
| Measurement | Timepoint | Mean (95% CI) | Mean (95% CI) |
| Capacitance | Baseline | 34.1 (32.4-35.2) | 34.1 (33.1-35.1) |
| | 1^{st} hour | 39.2 (38.3-40.1) | 35.1 (34.1-36.4) |
| | 3^{rd} hour | 40.1 (38.6-40.9) | 35.7 (34.2-36.8) |

As capacitance is an arbitrary value, numerical transformation to % change was performed to further assess the change as compared with baseline. Following were percentage change values in capacitance at 1 and 3 hours for the composition as well as control (Table 3 & FIG. 2).

TABLE 3
Percentage (%) change in capacitance at 1 and 3 hours

| | | Investigational | |
|---|---|---|---|
| | | Product | Control |
| Measurement | Timepoint | Mean (95% CI) | Mean (95% CI) |
| Capacitance | 1^{st} hour | 117.0 (114.6-121.1) | 102.4 (101.3-104.6) |
| (% change) | 3^{rd} hour | 118.0 (113.2-122.3) | 111.0 (105.4-116.7) |

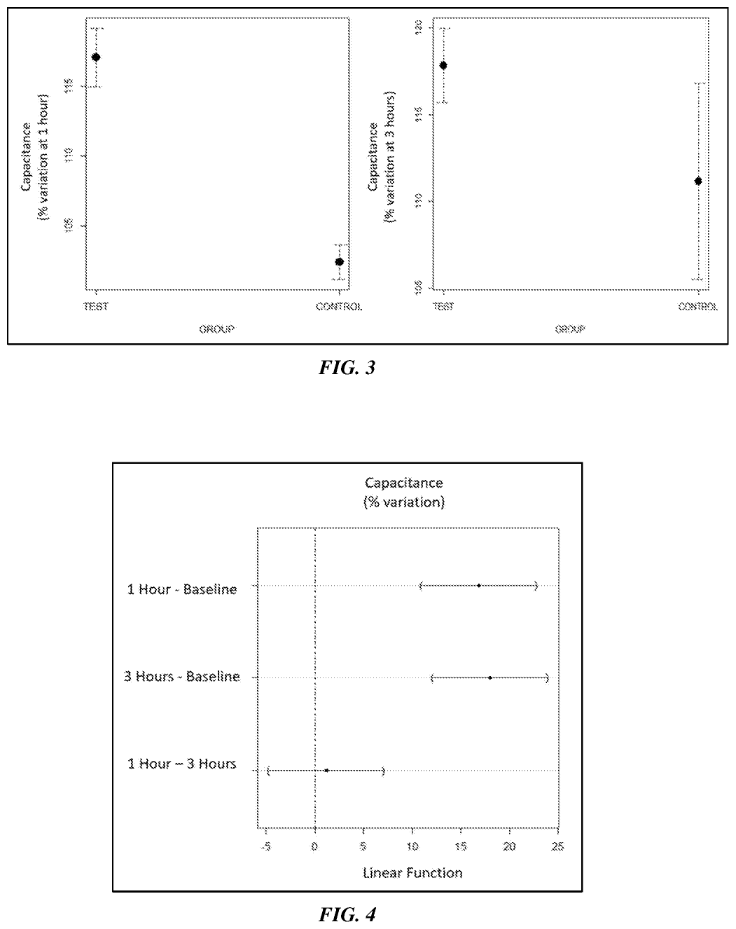

To assess the hypothesis that use of the investigational product produces changes in capacitance, and therefore is related to skin hydration, a Welch two sample t-test was performed on capacitance measurements between the composition and the control at 1 and 3 hours. Both tests were statistically significant (<0.001) confirming a significant variation of capacitance between the composition and control during the short-term period (FIG. 3).

To establish effect size, ANOVA and Cohen's d were used to compare results at 1 hour and at 3 hours with baseline data. Statistically significant difference in capacitance was observed by ANOVA between measurements at baseline, at 1 and 3 hours (p<0.00001) for the composition but not for the control. Multiple comparisons of means by Tukey contrasts showed that this difference is statistically significant when comparing baseline with 1 hour and baseline with 3 hours, but not 1 hour with 3 hours (Table 4 and FIG. 4).

TABLE 4
Tukey contrasts for capacitance at 1 and 3 hours

| Measurement | Comparison | Estimate | p value |
|---|---|---|---|
| Capacitance | Baseline-1 hour | 16.9 (11.0-22.8) | <0.00001 |
| (% change) | Baseline-3 hours | 18.0 (12.1-23.9) | <0.00001 |
| | 1 hour-3 hours | 1.17 (−4.7-7.0) | 0.858 |

Effect size estimation using Cohen's d shows a large effect (Table 5) at both comparisons previously reported as significant (i.e., comparing baseline with 1 hour and baseline with 3 hours).

TABLE 5
Effect size estimation for capacitance at 1 and 3 hours

| | | Cohen's d estimate |
|---|---|---|
| Measurement | Comparison | (lower-upper) |
| Capacitance | Baseline-1 hour | 1.6 (2.0-1.1) |
| (% change) | Baseline-3 hours | 1.7 (2.1-1.2) |

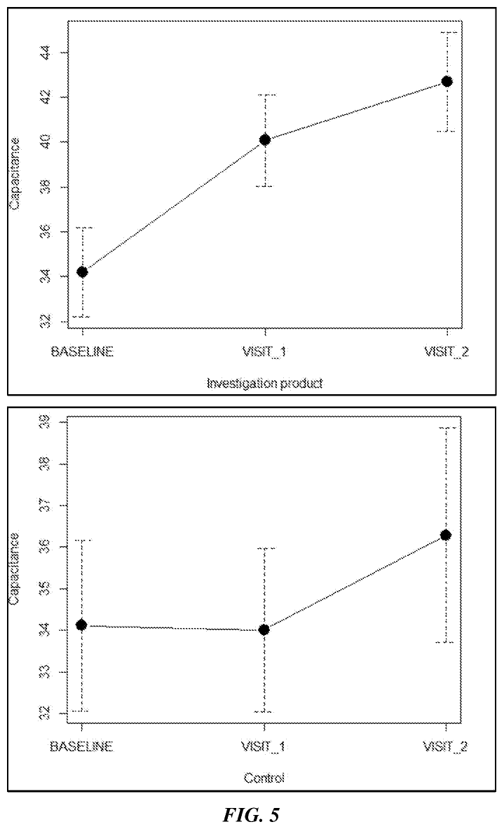

Capacitance measured at baseline and after repeated use was measured at 2 weeks and at 4 weeks (i.e., visit 1 and visit 2, respectively) for the composition and the control. The following values of capacitance at baseline, visit 1 and visit 2 after repeated application of composition versus control were obtained (Table 6 and FIG. 5).

TABLE 6
Mean capacitance and 95% confidence interval (CI)

| | | Investigational | |
|---|---|---|---|
| | | Product | Control |
| Measurement | Timepoint | Mean (95% CI) | Mean (95% CI) |
| Capacitance | Baseline | 34.1 (32.4-35.2) | 34.1 (33.1-35.1) |
| | Visit 1 | 40.0 (37.8-42.1) | 33.9 (32.2-35.7) |
| | 3^{rd} hour | 42.6 (40.2-44.7) | 36.3 (33.5-38.8) |

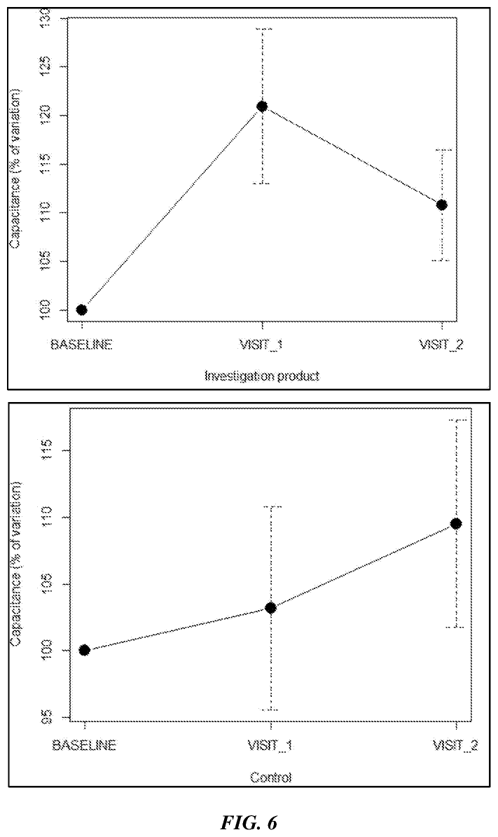

Following were percentage change values in capacitance at 2 and 4 weeks for the composition as well as control (Table 7 and FIG. 6).

TABLE 7
Percentage (%) change in capacitance
at 2 (visit 1) and 4 weeks (visit 2)

| | | Investigational | |
|---|---|---|---|
| | | Product | Control |
| Measurement | Timepoint | Mean (95% CI) | Mean (95% CI) |
| Capacitance | 2 weeks | 120.2 (112.6-129.1) | 102.5 (95.5-110.6) |
| (% change) | 4 weeks | 110.1 (105.1-116.3) | 109.5 (101.6-116.4) |

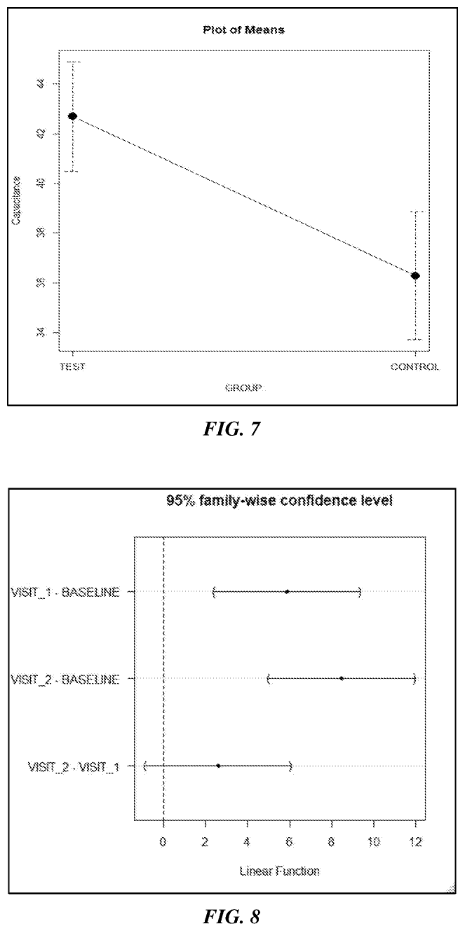

To assess the hypothesis that use of the composition produces changes in capacitance, and therefore is related to skin hydration, a Welch two sample t-test was performed on capacitance measurements between the investigational product and the control between base line and visit 2. Both tests were statistically significant (<0.001) confirming a significant variation of capacitance between the investigational product and control (FIG. 7).

As primary outcome was assessed as having significant variation, to establish effect size ANOVA and Cohen's d were used to compare results at 2 and 4 weeks with baseline data. Statistically significant difference in capacitance was observed by ANOVA between measurements at baseline and visit 1 as well as visit 2 (p<0.001) for the investigational product but not for the control. Multiple comparisons of means by Tukey contrasts showed that this difference is statistically significant when comparing baseline with visit 1 and baseline with visit 2, but not visit 1 with visit 2 (Table 8 and FIG. 8).

TABLE 8
Tukey contrasts for capacitance at 2
weeks (visit 1) and 4 weeks (visit 2)

| Measurement | Comparison | Estimate | p value |
|---|---|---|---|
| Capacitance | Baseline-Visit 1 | 5.8 (2.4-9.3) | <0.001 |
| (% change) | Baseline-Visit 2 | 8.4 (5.0-11.9) | <0.0001 |
| | Visit 1-Visit 2 | 2.5 (−0.8-6.0) | 0.18 |

Finally, effect size estimation using Cohen's d shows a large effect (Table 9) at the end of the long-term follow-up suggesting a large effect on capacitance after repeated use (i.e., comparing baseline with visit 2).

TABLE 9
Effect size estimation for capacitance at week 4 (visit 2)

| | | Cohen's d estimate |
|---|---|---|
| Measurement | Comparison | (lower-upper) |
| Capacitance | Baseline-Visit 2 | 1.16 (0.72-1.5) |
| (% change) | | |

Effects on Erythema

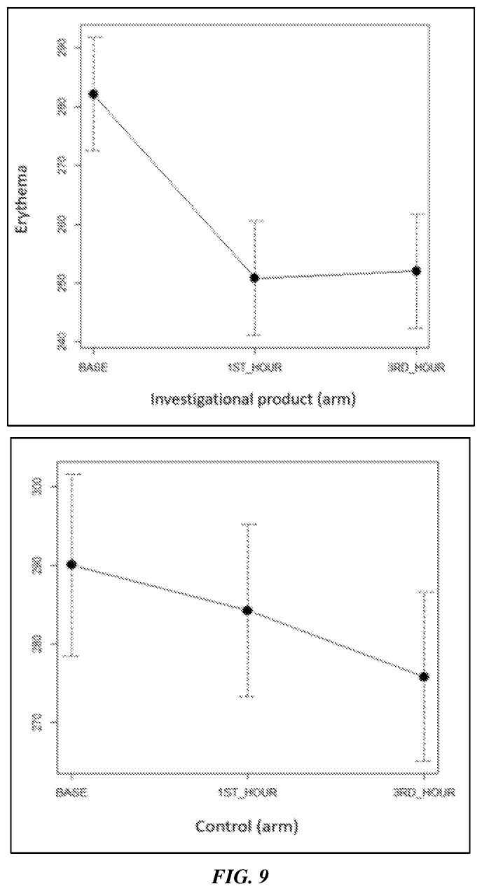

Results showed significant improvement in erythema on volar forearm in subjects treated with the composition versus control. Effects were assessed at baseline, at 1 hour and at 3 hours (Table 10 and FIG. 9).

TABLE 10
Mean erythema on volar forearm in subjects
treated with the composition versus control

| | | Investigational | |
|---|---|---|---|
| | | product | Control |
| Measurement | Timepoint | Mean (95% CI) | Mean (95% CI) |
| Erythema | Baseline | 282.1 (273.3-292.5) | 290.0 (276.7-303.4) |
| | 1^{st} hour | 250.8 (241.6-260.2) | 284.2 (273.3-294.2) |
| | 3^{rd} hour | 253.0 (242.6-262.8) | 277.4 (266.6-289.1) |

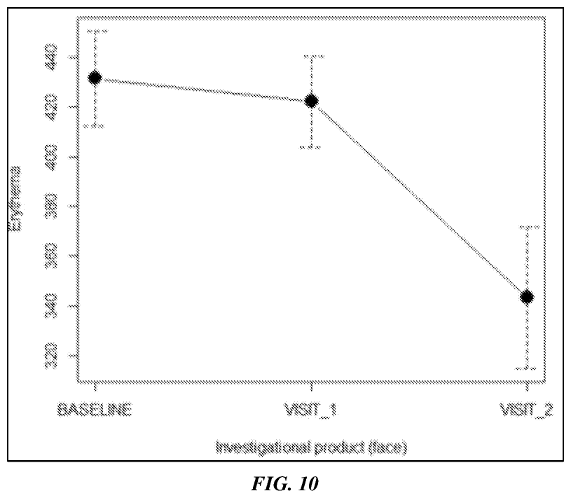

Significant improvement was observed in facial erythema at week 2 (visit 1) and week 4 (visit 2) by repeated use of the composition (FIG. 10).

These results were consistent with a large effect on hydration as measured by capacitance after single (i.e., short-term) and repeated (i.e., long-term) use, additionally, a significant and large effect on erythema on the face was observed. These results support a large effect of the investigational product on hydration and subliminal inflammation. No adverse events were reported during the study.

While the foregoing has presented specific embodiments of the present disclosure, it is to be understood that these embodiments have been presented by way of example only. It is expected that others skilled in the art will perceive variations which, while varying from the foregoing, do not depart from the spirit and scope of the disclosure herein.

It must be noted that as used in this specification and the appended claims, the singular forms “a,” “an,” and “the” include plural reference unless the context clearly dictates otherwise. Unless defined otherwise all technical and scientific terms used herein have the same meaning as commonly understood to one of ordinary skill in the art to which this disclosure belongs.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to encompass the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items.

As used herein, whether in the specification or the appended claims, the transitional terms “comprising”, “including”, “having”, “containing”, “involving”, and the like are to be understood as being inclusive or open-ended (i.e., to mean including but not limited to), and they do not exclude unrecited elements, materials or method steps. Only the transitional phrases “consisting of” and “consisting essentially of”, respectively, are closed or semi-closed transitional phrases with respect to claims and exemplary embodiment paragraphs herein. The transitional phrase “consisting of” excludes any element, step, or ingredient which is not specifically recited. The transitional phrase “consisting essentially of” limits the scope to the specified elements, materials or steps and to those that do not materially affect the basic characteristic(s) of the disclosure herein.

1. A topical skin care composition comprising:
a. a cannabinoid at 0.01-10% (w/w),
b. a skin conditioning agent at 0.01-5% (w/w),
c. a skin soothing agent at 1-5% (w/w),
d. an occlusive at 0.01-5% (w/w),
e. an emulsifier at 1-10% (w/w),
f. water to make up 100% by weight,
wherein the topical skin care composition comprises less than 20% (w/w) of fatty alcohol.

2. The topical skin care composition of claim 1, wherein the topical skin care composition comprises no more than 10% (w/w) of fatty alcohol.

3. The topical skin care composition of claim 1, wherein the topical skin care composition comprises no more than 5% (w/w) of fatty alcohol.

4. A topical skin care composition comprising:
a. a cannabinoid at 0.01-10% (w/w),
b. a skin conditioning agent at 0.01-5% (w/w),
c. a skin soothing agent at 1-5% (w/w),
d. an occlusive at 0.01-5% (w/w),
e. an emulsifier at 1-10% (w/w),
f. water no less than 51% (w/w).

5. The topical skin care composition of any of claims 1 to 4, wherein the skin conditioning agent is colloidal oatmeal, the skin soothing agent is oat kernel extract, the occlusive is hemp seed oil and the emulsifier is glyceryl stearate.

6. The topical skin care composition of any one of claims 1 to 5, wherein the cannabinoid is phytocannabinoid.

7. The topical skin care composition of claim 6, wherein the phytocannabinoid is tetrahydrocannabinol (THC), tetrahydrocannabinolic acid (THCA), cannabidiol (CBD), cannabidiolic acid (CBDA), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigervarin (CBGV), cannabigerol monomethyl ether (CBGM), cannabielsoin (CBE), cannabicitran (CBT), or any combination thereof.

8. The topical skin care composition of claim 7, wherein the cannabinoid is cannabidiol (CBD).

9. The topical skin care composition of any one of claims 1 to 8, which has a pH of 5-7.

10. The topical skin care composition of any one of claims 1 to 9, wherein the composition further comprises one or more of:
a. a first surfactant at 1-10% (w/w),
b. a second surfactant at 1-5% (w/w),
c. a first emollient at 1-15% (w/w),
d. a second emollient at 0.01-5% (w/w),
e. a humectant at 0.01-5% (w/w),
f. a penetration enhancer at 1-10% (w/w),
g. an opacifying agent at 0.01-3% (w/w),
h. a stabilizer at 0.01-5% (w/w),
i. a preservative at 0.01-3% (w/w),
j. pH adjusting agents in a quantity sufficient for the composition to maintain a pH of 5-7, and
k. water to make up 100% by weight.

11. The topical skin care composition of claim 10, wherein the first surfactant is cetostearyl alcohol, the second surfactant is Tween 60, the first emollient is white petrolatum, the second emollient is dimethicone, the humectant is glycerin, the penetration enhancer is ethoxydiglycol, the opacifying agent is titanium (IV) oxide, the stabilizer is disodium salt of ethylenediaminetetraacetic acid (EDTA), the preservative is phenoxyethanol SA, pH adjusting agents are trisodium citrate dihydrate and citric acid monohydrate.

12. The topical skin care composition of any one of claims 1 to 11, wherein the composition is a cream, ointment, gel, lotion, liquid, solution, spray, aerosol, any other dosage forms suitable for topical application, or any combination thereof.

13. The topical skin care composition of claim 12, wherein the composition is a lotion.

14. A lotion composition comprising:
a. a cannabinoid at 0.01-10% (w/w),
b. a skin conditioning agent at 0.01-5% (w/w),
c. an emulsifying agent 1-10% (w/w),
d. an occlusive at 0.01-5% (w/w), and
e. water to make up 100% by weight.

15. The lotion of claim 14, wherein the composition has a viscosity in a range of about 5,000 to about 100,000 cps.

16. A method comprising applying the topical skin care composition of any one of claims 1 to 15 to the skin of a subject.

17. The method of claim 16, wherein the skin of the subject is affected by a skin disease or condition.

18. The method of claim 17, wherein the skin disease or condition is: contact eczema/dermatitis, atopic eczema/dermatitis, seborrhoeic eczema/dermatitis, stasis eczema/dermatitis, nummular eczema/dermatitis, lichen simplex chronicus, dyshidrotic eczema/dermatitis, erythema, irritation, pruritus, dried skin, cracked skin, wrinkles and rosacea.

19. Use of the topical skin care formulation of any one of claims 1 to 15 for the treatment of a skin disease or condition in a subject.

20. The use of claim 19, wherein the skin disease or condition is: contact eczema/dermatitis, atopic eczema/dermatitis, seborrhoeic eczema/dermatitis, stasis eczema/dermatitis, nummular eczema/dermatitis, lichen simplex chronicus, dyshidrotic eczema/dermatitis, erythema, irritation, pruritus, dried skin, cracked skin, wrinkles and rosacea.
